(19) United States
(12) Patent Application Publication
(10) Pub. No.: US 2016/0022743 A1
(43) Pub. Date: Jan. 28, 2016

(54) HUMAN VERY SMALL EMBRYONIC-LIKE (VSEL) STEM CELLS FOR TREATMENT OF OCULAR DISEASE

(71) Applicants: NEOSTEM, INC., New York, NY (US); THE SCHEPENS EYE RESEARCH INSTITUTE, INC., Boston, MA (US)

(72) Inventors: Kameran LASHKARI, Boston, MA (US); Michael YOUNG, Essex, MA (US); Sarah EMINLI-MEISSNER, Cambridge, MA (US); Wayne MARASCO, Wellesley, MA (US); Robert PRETI, Ridgefield, CT (US)

(73) Assignee: The Schepens Eye Research Institute, Inc., Boston, MA (US)

(21) Appl. No.: 14/404,321

(22) PCT Filed: May 31, 2013
(86) PCT No.: PCT/US2013/043797

§ 371 Date: Nov. 26, 2014

Related U.S. Application Data
(60) Provisional application No. 61/654,002, filed on May 31, 2012.

(51) Int. Cl. A61K 35/545; A61K 9/00; A61K 45/06

(52) U.S. Cl. CPC A61K 35/545; A61K 45/06; A61K 9/0048

(57) ABSTRACT

The present invention relates to use of very small embryonic-like (VSEL) stem cells in therapies for ocular disease involving retinal degeneration or dysfunction. The invention also included pharmaceutical compositions made with VSELs which may be used to restore lost vision or reduce or halt vision loss due to diseases or disorders of the retina, or other diseases or retinal injuries that would benefit from stem cell replacement therapy.

### CROSS-REFERENCE TO RELATED APPLICATIONS

This application claims priority to U.S. Application No. 61/654,002, filed May 31, 2012, which is incorporated herein by reference in its entirety.

### FIELD OF THE INVENTION

The present invention relates to use of very small embryonic-like (VSEL) stem cells in therapies for ocular disease involving retinal degeneration or dysfunction. The invention also included pharmaceutical compositions made with VSELs which may be used to restore lost vision or reduce or halt vision loss due to diseases or disorders of the retina, or other diseases or retinal injuries that would benefit from stem cell replacement therapy.

### BACKGROUND OF THE INVENTION

Age-related macular degeneration (AMD) is one of the leading causes of incurable blindness in the western world. Atrophic (dry) AMD, the most prevalent form, accounts for 90% of cases, and is characterized by progressive deposition of debris under the retinal pigment epithelium (RPE), a highly specialized tissue that supports and protects the light sensitive photoreceptors of the outer neural retina, leading to their degeneration. Cell transplantation (RPE, photoreceptor precursors or cells with neuroprotective abilities) could be used to restore sight in advanced AMD by replenishing the subretinal anatomy and re-establishing the functional relationship between RPE and photoreceptors.

Retinitis pigmentosa includes inherited retinal disorders characterized by progressive degeneration of photoreceptors and RPE. Currently, there is no therapy that stops progression of the disease or restores vision. Another inherited disorder is Stargardt's disease, affecting children primarily between 6 and 12 years of age, which results in rapid and severe central vision loss.

Stem cell-based therapy is being pursued for treatment of retinal degenerative disease. Retinal stem cells have been isolated from several mammalian species, including humans. However, transplantation of these cells was minimally successful due to the limited ability of the cells to migrate and integrate into the host retina. Bone marrow-derived stem cells may be an alternative, but bone marrow contains several types of pluripotent/multipotent cells, including hematopoietic stem cells, mesenchymal stem cells, and a heterogeneous population of non-hematopoietic cells that differentiate into mesenchymal tissues but possibly into other tissue types. It has also been reported that bone marrow contains tissue-committed stem cells.

Very Small Embryonic-like Stem cells (VSELs) are pluripotent cells found in human bone marrow and in adult peripheral blood. VSELs are small in size, are lin⁻ and CD45⁻ cells, express CD133, CD34, CXCR4, and other components characteristic of embryonic stem cells such as SSEA-4, Oct-4, Rev-1, and Nanog.

### SUMMARY OF THE INVENTION

To demonstrate the regenerative potential of VSELs in the retina, PKH26-labeled enriched human VSELs were transplanted into the mouse eye—both by injecting the cells into the vitreous space, and by injecting the cells subretinally in a SCID mouse model of retinal detachment. The ability of human VSELs to engraft, survive and differentiate into retinal or neuroectodermal cells in the mouse retina was assessed. At 2 and 4 weeks after transplantation, subretinally and intravitreally injected human VSELs were able to engraft, survive and migrate within the retina. Furthermore, immunohistochemistry analysis revealed that subretinally transplanted cells differentiate and express markers of retinal stem and developing progenitor cells such as nestin and PAX6, of neuro-ectodermal cells such as MAP2 and beta-3-tubulin, and the early photoreceptor marker recoverin. These studies indicate that human VSELs can engraft, migrate and differentiate along the retinal lineage.

The invention relates to methods and compositions for treating damage or an injury to ocular tissue. According to the invention, a composition comprising an effective amount of very small embryonic like stem cells (VSELs) is administered to the tissue, wherein the VSELs repair or regenerate the retinal tissue. Methods of mobilizing, collecting, and purifying VSELs have been described.

Thus, the invention provides a method for treating or ameliorating a retinal disease of a mammal comprising administering an effective amount of a composition comprising very small embryonic-like stem cells (VSELs) into an eye of the mammal, where they migrate and differentiation along retinal lineages. In certain embodiments, VSELS comprise CD45⁻/lin⁻/CD34⁺, or CD45⁻/lin⁻/CD133⁺, or CD45⁻/lin⁻/CD34⁺/CD133⁺, and express one or more of SSEA-4, Oct-4, Rev-1, and Nanog, and are further described herein.

According to the invention, VSELs are administered to the eye of a mammal, including but not limited to, subretinally or intravitreally.

Ocular damage or injury treated according to the invention includes retinal disease, such as, without limitation, macular degeneration and retinitis pigmentosa.

VSELs administered according to the invention include autologous VSELs and allogeneic VSELs. The VSELs can be from any mammal, including mouse. In a preferred embodiment, the VSELs are human VSELs. The invention also includes xenogenic transplantation such as human VSELs into an animal model of human ocular disease.

In certain embodiments, at least a portion of the administered VSELs engraft and express one or more markers of neural or retinal differentiation such as Nestin, Pax6, rhodopsin, recoverin, β3-tubulin and MAP2.

The invention also provides for administering with VSELs, an agent that stimulates differentiation of said VSELs. For example, such an agent can stimulate differentiation into RPEs, photoreceptors cells, or neurons.

In certain embodiments, the VSELs differentiate at the site of repair. In certain embodiments, the VSELs are treated or selected to be differentiated towards a desired lineage. For example, in one embodiment, the VSELs are differentiated into cells that express a marker of RPE cells, for example one or more of RPE65, bestrophin, CRALBP, and PEDF, prior to administering to the mammal.

In certain embodiments, the VSELs are administered in a suspension or incorporated into a matrix, or scaffold, which may be biodegradable. In certain embodiments, the matrix is selected to elicit differentiation of the VSELs towards the desired tissue type. In certain embodiments, the VSELs are administered with an agent or growth factor that promotes differentiation towards the desired tissue type.

The number of VSELs in the composition that is employed can be related to the volume or the surface area of the ocular defect to be treated. In an embodiment of the invention, the composition comprises from about 50 to about 50,000 VSELs. In another embodiment, the composition comprises about 200 to about 10,000 VSELs. In an embodiment of the invention, the composition comprises from about 500 to about 5,000 VSELs. In other embodiments, the composition comprises from 20 to 200, or from 200 to 1000, or from 1000 to 5,000, or from 50,000 to 50,000 VSELs.

In certain embodiments, the VSELs are provided in a composition that comprises other nucleated cells. In certain such embodiments, the cells of the composition are at least about 50% VSELs. In other embodiments, at least about 70% of the cells of the composition are VSELs. In additional embodiments, at least about 90% or at least about 95% of the cells of the composition are VSELs.

### DESCRIPTION OF THE FIGURES

FIG. 1. Injection of cells into the vitreous cavity or subretinal space. Exemplary methods of VSEL administration are depicted. The photoreceptor layer is also called the Outer Nuclear Layer (ONL). (Reproduced from K. Palczewski, FASEB J 2011; 25:439-443)

FIG. 2. PKH-26⁺ cell-counts four weeks after intravitreal injection. a) Cy3 channel (top), FITC channel (middle) and merged (bottom) images indicate that positive staining in Cy3 channel (arrows) is due to PKH-26 labeled cells and not auto-fluorescence. b) Representative cryosections with PKH-26⁺ cell counts shown. c) Cy3 channel (top) and DAPI (center) merged with FITC channel (beta-III-tubulin, which stains the nerve fiber layer) indicate that the PKH-26⁺ cells co-localize with cell nuclei along the inner limiting membrane and tend not to migrate into the retina.

FIG. 3. PKH-26⁺ cells migrate into the retina following subretinal injection. GCL: ganglion cell layer, INL: inner nuclear layer, ONL: outer nuclear layer, RPE: retinal pigment epithelium. a) PKH-26⁺ cells (arrows) identified within the outer nuclear layer of the retina 2 weeks following injection into the sub-retinal space. b) PKH-26⁺ cells identified in the subretinal space, outer nuclear layer and inner nuclear layer.

FIG. 4. Some transplanted cells stain for markers of retinal differentiation. GCL: ganglion cell layer, INL: inner nuclear layer, ONL: outer nuclear layer, RPE: retinal pigment epithelium. a) Staining with antibody against human SRP14 shows double staining for PKH-26+ cells (arrows), plus additional cells that may have lost the PKH-26 marker. Antibody to human nuclear antigen (HuN) did not stain in this experiment. b) Anti-Nestin antibody identifies PHK-26⁺/SRP14⁺/Nestin⁺ cells (arrow). c) Anti-PAX6 antibody identifies rare PHK-26⁺/SRP14⁺/PAX6⁺ cells (arrows). d) Anti-Recoverin antibody identifies PHK-26⁺/Recoverin⁺ cells (arrows). e) Anti-b3-tubulin antibody identifies rare PHK-26⁺/SRP14⁺/b3-tubulin⁺ cells (arrows). f) Anti-MAP2 antibody identifies PHK-26⁺/SRP14⁺/MAP2⁺ cells (arrows).

### DETAILED DESCRIPTION

The present invention provides therapeutic compositions and methods for treating or ameliorating retinal degeneration. According to the invention, very small embryonic-like stem cells (VSELs) are used in methods that preserve or restore retinal tissue and function. More particularly, VSELs are introduced into retinal tissue where they migrate and restore eye cells, including, but not limited to, retinal cells such as rods and cones, and neural cells such as amacrine cells (interneurons in the retina) and retinal ganglion cells. Markers characteristic of neural differentiation include, without limitation, nestin and MAP2 (microtubule assembly, which is an essential step in neurogenesis). Non-limiting markers of photoreceptors include recoverin, rhodopsin, Crx, and Nrl.

The term “very small embryonic-like stem cell” is also referred to herein as “VSEL stem cell” and refers to pluripotent stem cells. In some embodiments, the VSEL stem cells (“VSELs”) are human VSELs and may be characterized as Lin⁻, CD45⁻, and CD34⁺. In some embodiments, the VSELs are human VSELs and may be characterized as Lin⁻, CD45⁻, and CD133⁺. In some embodiments, the VSELs are human VSELs and may be characterized as Lin⁻, CD45⁻, and CXCR4⁺. In some embodiments, the VSELs are human VSELs and may be characterized as Lin⁻, CD45⁻, CXCR4⁺, CD133⁺, and CD34⁺. In some embodiments, the VSELs are human VSELs and may be characterized as Lin⁻, CD45⁻, CD133⁺, and CD34⁺. In some embodiments, human VSELs express at least one of SSEA-4, Oct-4, Rex-1, and Nanog. With respect to stem cell markers, mouse VSELs express at least one of SSEA-1, Oct-4, Rex-1, and Nanog. VSELs may also be characterized as possessing large nuclei surrounded by a narrow rim of cytoplasm, and contain embryonic-type unorganized chromatin. VSELs also have high telomerase activity. In some embodiments, the VSELs are human VSELs and may be characterized as Lin⁻, CD45⁻, CXCR4⁺, CD133⁺, Oct 4+, SSEA4⁺, and CD34⁺. In some embodiments, the human VSELs may be less primitive and may be characterized as Lin⁻, CD45⁻, CXCR4⁺, CD133⁻, and CD34⁺. In some embodiments, the human VSELs may be enriched for pluripotent embryonic transcription factors, e.g., Oct-4, Sox2, and Nanog. In some embodiments, the human VSELs may have a diameter of 4-5 μm, 4-6 μm, 4-7 μm, 5-6 μm, 5-8 μm, 6-9 μm, or 7-10 μm. VSELs administered according to the invention can be collected and enriched or purified and used directly, or frozen for later use. Autologous or allogeneic VSELs can be administered according to the invention. Further, the VSELs may be engineered.

VSEL stem cells and extracellular vesicles with similar surface markers can be obtained and isolated or purified by a variety of procedures, for example on the basis of their size, density, and staining characteristics (e.g., surface markers, lack of staining by nuclear dyes). In one embodiment, the VSELs are obtained from peripheral blood by apheresis. In an embodiment of the invention, the VSELs are obtained from mobilized peripheral blood. In another embodiment, the VSELs are obtained from cord blood. In another embodiment, the VSELs are obtained from bone marrow. In another embodiment, the VSELs are obtained from spleen. In another embodiment, the VSELs are obtained from adipose tissue. In another embodiment, the VSELs are obtained from adult tissue. In some embodiments, the VSELs are co-cultured with C2C12 feeder layers prior to use. In some embodiments, the VSELs are propagated as embryoid body-like spheres.

WO/2011/069117 describes a method of isolation of stem cell populations from peripheral blood using sized-based separation. Fresh apheresed cells are lysed with 1×BD Pharm Lyse Buffer, in a ratio of approximately 1:10 (vol/vol) to remove red blood cells. After washing, cells are counted, and 2-2.5×10¹⁰ total nucleated cells are loaded onto the ELUTRA® Cell Separation System (CaridianBCT) at a concentration of 1×10⁸ cells/ml. Cells are then collected in 900 ml PBS+0.5% HSA media in each bag at different flow rates. Typically, six fractions are collected with a centrifugation speed of 2400 rpm. Finally, cells from all fractions are transferred into tubes and spun down at 600×g for 15 minutes. Size characteristics of the fractions are confirmed by evaluating SSC and FSC. As disclosed therein, certain fractions are highly enriched in VSELs and can be used to provide populations of VSELs for clinical applications. The procedure can be adapted to other equipment. The populations may be further purified by FACS.

Using methods that separate cells based on size or density such as differential centrifugation, percoll gradient centrifugation, and counterflow centrifugal elutriation, it was observed that the Lin⁻CD45⁻CD34/133⁺ events fall into two separate populations with different physical characteristics—a major population (approximately 98% of Lin⁻CD45⁻CD34/133⁺ events) of objects that are very small (<4 μm), very light, and stain negatively or dimly with the nuclear dye DRAQ5, and a minor population that is larger (5-10 μm), heavier, and that stains brightly with DRAQ5. FACS sorting of the two populations followed by cytospin and diff-quick stain showed that the minor population consists of small nucleated cells, whereas the major population consists of membrane-bound objects that do not have a cell nucleus. By light microscopy and transmission electron microscopy these objects have the appearance of extracellular vesicles. Although most are roughly the size of platelets, their morphologic appearance is quite different from platelets. The two populations of Lin⁻CD45⁻CD34/133⁺ events are also found in umbilical cord blood, although at different frequencies than in mobilized adult blood (1 for every 5 hematopoietic progenitors, with 94% of the events being DRAQ5⁻). Accordingly, when VSELs are isolated or purified by flow cytometry, a nuclear marker such as DRAQ5 can be useful to quantify nucleated VSELs among other cell-like objects cells having VSEL markers or characteristics. As with the DRAQ5⁺ nucleated VSELs, the enucleated particles which express markers of VSELs may also be purified and used in therapies to repair damaged or injured tissues in human subjects.

According to the invention, VSEL subpopulations may be selected for expression of markers indicating differentiation towards a desired tissue type. Also, VSELs may be treated or cultured prior to administration to induce differentiation towards a desired tissue type.

The invention provides therapeutic methods for retinal diseases and dysfunctions. Retinal dysfunction encompasses any lack or loss of any lack or loss of normal retinal function, whether due to disease, mechanical, or chemical injury, or a degenerative or pathological process involving the recipient's retina. The VSELs may be injected or otherwise placed in a retinal site, the subretinal space, vitreal cavity (including injection or other introduction into the vitreous of the vitreal cavity), or the optic nerve, according to techniques known in the art. This includes the use of a biodegradable substrates as a carrier for the VSELs.

In certain embodiments, the invention provides for administration of autologous stem cells. In certain embodiments, the invention provides for administration of allogeneic stem cells. In this regard, certain tissues of the eye, including the vitreous cavity, pigment epithelium, retina, and subretinal space, are immune privileged, allowing for allogeneic tissue grafts. In such embodiments, VSELs collected from a donor may be administered to a recipient. To further minimize or prevent immune rejection of the tissue graft, the donor and recipient can be matched, and/or immunosuppressive agents can be administered. In certain embodiments, including, but not limited to animal models of ocular disease, the invention provides for xenografts.

The term “regeneration” as used herein refers to reproduction or reconstitution of a lost or injured part.

The term “repair” as used herein refers to restoration of diseased or damaged tissues naturally by healing processes or artificially.

The term “therapeutically effective” as used herein refers to the amount of the autologous stem cell product comprising human very small embryonic like stem cells (VSELs) that results in a therapeutic or beneficial effect following its administration to a subject. The therapeutic effect may be curing, minimizing, preventing or ameliorating a disease or disorder, or may have any other beneficial effect. The concentration of the substance is selected so as to exert its therapeutic effect, but low enough to avoid significant side effects within the scope and sound judgment of the physician. The effective amount of the autologous stem cell product may vary with the age and physical condition of the biological subject being treated, the severity of the condition, the duration of the treatment, the nature of concurrent therapy, the timing of the infusion, the specific compound, composition or other active ingredient employed, the particular carrier utilized, and like factors.

A skilled artisan may determine a therapeutically effective amount of the autologous stem cell product comprising human very small embryonic like stem cells (VSELs) by determining the dose in a dosage unit (meaning unit of use) that elicits a given intensity of effect, hereinafter referred to as the “unit dose.” The term “dose-intensity relationship” refers to the manner in which the intensity of effect in an individual recipient relates to dose. The intensity of effect generally designated is 50% of maximum intensity. The corresponding dose is called the 50% effective dose or individual ED₅₀. The use of the term “individual” distinguishes the ED₅₀ based on the intensity of effect as used herein from the median effective dose, also abbreviated ED₅₀, determined from frequency of response data in a population. “Efficacy” as used herein refers to the property of the compositions of the described invention to achieve the desired response, and “maximum efficacy” refers to the maximum achievable effect. The amount of the autologous stem cell product that will be effective in the treatment of a particular disorder or condition will depend on the nature of the disorder or condition, and may be determined by standard clinical techniques. (See, for example, Goodman and Gilman's THE PHARMACOLOGICAL BASIS OF THERAPEUTICS, Joel G. Harman, Lee E. Limbird, Eds.; McGraw Hill, New York, 2001: THE PHYSICIAN'S DESK REFERENCE, Medical Economics Company, Inc., Oradell, N. J., 1995; and DRUG FACTS AND COMPARISONS, FACTS AND COMPARISONS, INC., St. Louis, Mo., 1993), each of which is incorporated by reference herein. The precise dose to be employed in the formulations of the described invention also will depend on the route of administration and the seriousness of the disease or disorder, and should be decided according to the judgment of the practitioner and each subject's circumstances.

As used herein the terms “treat” or “treating” are used interchangeably to include abrogating, substantially inhibiting, slowing or reversing the progression of a condition, substantially ameliorating clinical or aesthetical symptoms of a condition, substantially preventing the appearance of clinical or aesthetical symptoms of a condition, and protecting from harmful or annoying stimuli. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

In some embodiments, the dysfunctional retina is the result of age-related macular degeneration, early onset macular degeneration, Usher Syndrome, retinitis pigmentosa, choroideremia, cone dystrophy, cone-rod dystrophy, rod-cone dystrophy, Leber's congential amaurosis, congential stationary night blindness, Sticklers Syndrome, colobomas, vitreoretinal dysplasia, achromatopsia, or optic nerve hypoplasia.

The methods can be used to treat a mammalian recipient suffering from a lack or diminution of photoreceptor cell function. Examples of retinal dysfunction that can be treated by the adult retinal stem cell lines and methods of the invention include but are not limited to: photoreceptor degeneration (as occurs in, e.g., hereditary or acquired retinitis pigmentosa, cone dystrophies, cone-rod and/or rod-cone dystrophies, and macular degeneration, including age-related and early onset macular degeneration); retinal detachment and retinal trauma; photic lesions caused by laser or sunlight; a macular hole; a macular edema; night blindness and color blindness; ischemic retinopathy as caused by diabetes or vascular occlusion; retinopathy due to prematurity/premature birth; infectious conditions, such as, e.g., CMV (cytomegalovirus) retinitis, herpes type 1 retinitis, Ebstein-Barr virus retinitis, toxoplasmosis, rubella and pox virus; inflammatory conditions, such as the uveitidies, multifocal choroiditis and uveitis, birdshot chorioretinopathy, collagen vascular diseases affecting the posterior segment of the eye, including Wegener's granulomatosis, uveitis associated with systemic lupus erythematosus, uveitis associated with polyarteritis nodosa, peripheral or intermediate uveitis, chronic central serous chorioretinopathy, and myopic choroidal neovascular membranes and scars. Inflammatory disorders also include Behcet syndrome, intermediate uveitis (pars planitis), masquerade syndromes, peripheral uveitis, ocular syphilis, ocular tuberculosis, viral-related chorioretinitis (ARN) syndrome, HIV-related uveitis, progressive outer retinal necrosis syndrome, sympathetic ophthalmia, white dot syndromes, presumed ocular histoplasmosis syndrome, acute macular neuroretinopathy, diffuse unilateral subacute neuroretinitis, ophthalmomyiasis, serpiginous choroidopathy, panuveitis, birdshot retinochoroidopathy, and uveitis associated with disorders such as juvenile rheumatoid arthritis, Kawasaki syndrome, multiple sclerosis, sarcoidosis, toxocariasis, toxoplasmosis, Vogt-Koyanagi-Harada (VKH), and HLA-B27 seropositive spondylopathy syndromes.

Other disorders include tumors, such as retinoblastoma and ocular melanoma. Additionally, the VSELs can be used for replacement of inner retinal neurons, which are affected in ocular neuropathies including glaucoma, traumatic optic neuropathy, degenerative optic neuropathy, ischemic optic neuropathy, optic neuropathy from multiple sclerosis, and radiation optic neuropathy and retinopathy.

The methods can also be used to treat optic nerve diseases such as optic atrophy, ischemic optic neuropathy, diabetes induced optic atrophy, optic nerve hypoplasia, morning glory syndrome, Graves ophthalmopathy, optic neuritis, cytomegalovirus neuritis, arteritic optic neuropathy, compressive neuropathy, diabetic neuropathy, giant cell arteritis, infiltrative neuropathy, nutriotional, ischemic neuropathy, retrobulbar optic neuritis, retrobulbar ischemic neuropathy, toxic neuropathy, traumatic neuropathy; optic nerve diseases resulting from causes such as syphilis, Lyme disease, toxoplasmosis, cat scratch disease, systemic lupus erythematosus, paraneoplastic syndrome, multiple sclerosis, and autoimmune disease; degenerative optic diseases such as age-related macular degeneration, early onset macular degeneration, Usher Syndrome, retinitis pigmentosa, cone-road dystrophy, and choroideremia; and congenital optical diseases such as Leber's congential amaurosis, congential stationary night blindness, and optic nerve hypoplasia.

According to the invention, VSELs are introduced into a retinal site, a subretinal space, an optic nerve, or a vitreal cavity in amounts effective to treat or ameliorate a retinal disease or dysfunction. The amount of a VSEL composition will depend on the disease to be treated, and the degree to which VSELs are purified. For instance as provided above, compositions comprising VSELs purified on the basis of size and surface markers may contain enucleated cell-like vesicles as well as VSELs. Such vesicles, originating from intact cells, may contain biologically active components, but would not be capable of migration or differentiation. On the other hand, an elutriation procedure that separates cells on the basis of size and density would be expected to provide enriched VSEL compositions free from such enucleated cell-like vesicles.

In certain embodiments, the VSELs are provided in a composition that comprises other nucleated cells. In certain such embodiments, the cells of the composition are at least 50% VSELs. In other embodiments, at least 70% of the cells of the composition are VSELs. In additional embodiments, at least 90% or at least 95% of the cells of the composition are VSELs.

As exemplified herein, for intravitreal injection in mice, approximately 2,000 FACS-purified VSELs were injected into the vitreal space. For subretinal injection studies, approximately 10,000 cells (in preparations that ranged from 0.1% to 11% pure) were injected into the subretinal space. The VSELs were in a volume sufficient to create a subretinal detachment as the site of injection. It will be understood that when VSELs are administered according to the invention, such retinal damage can be avoided by reducing the volume of injected VSELs or by distributing the injected VSELs at multiple injection sites. It will be within the discretion of a skilled user to adjust the number of cells, purity of cell, volume of cells, and injection technique according to the retinal disorder being treated.

Accordingly, in an embodiment of the invention, an injectable VSEL composition can comprises from 50 to 50,000 VSELs. In another embodiment, the composition can comprise from 200 to 10,000 VSELs. In another embodiment of the invention, the composition comprises from 500 to 5,000 VSELs.

In certain embodiments of the invention, the number of VSELs administered is from 20 to 200. In other embodiments of the invention, the number of VSELs administered is from 200 to 1,000. In still other embodiments of the invention, the number of VSELs administered is from 1,000 to 5,000. In further embodiments of the invention, the number of VSELs administered is from 5,000 to 50,000.

Administration of the cells or compositions can be effected in one dose, continuously or intermittently throughout the course of treatment. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the invention can be administered in combination with other treatments.

In using the VSELs to treat retinal dysfunction, one can, in conjunction with introducing the VSELs into a recipient's eye, administer a substance that stimulates differentiation into photoreceptor cells or other retinal cell types (e.g., bipolar cells, ganglion cells, horizontal cells, amacrine cells, Mueller cells). When VSELs are introduced to treat a neural dysfunction of the eye, one can also utilize a substance (or combination of substances) that stimulates differentiation of stem cells into neurons or astrocytes.

In some embodiments, a therapeutic method of the invention further comprises administering to the recipient, a substance that stimulates differentiation of the VSELs into RPE cells. In some embodiments, a therapeutic method of the invention further comprises administering to the recipient, a substance that stimulates differentiation of the VSELs into photoreceptors cells. In some embodiments, a therapeutic method of the invention further comprises administering to the mammalian recipient, a substance that stimulates differentiation of the VSELs into neurons.

The invention also provides means to study and develop treatment for various ocular diseases, disorders, and injuries, particularly those involving retinal and neural retinal tissue.

The cellular origin of the retina is ectodermal, with retinal progenitor cells eventually forming two layers, RPEs originating from the outer layer, and the neural retina from the inner layer. The presently disclosed subject matter also provides methods for differentiating a VSEL stem cell towards a cell type of interest. The methods comprise culturing VSELs in a culture medium comprising a differentiation-inducing amount of one or more factors that induce differentiation of the VSEL stem cells into the cell type of interest until the cell type of interest appears in the culture.

In some embodiments, the cell type of interest is a neuronal cell or a derivative thereof, such as an astrocyte, a glial cell, or a neuron. In some embodiments, the neuronal cell or derivative thereof expresses one or more of GFAP, nestin, β III tubulin, Olig1, and Olig2. In some embodiments, VSELs are cultured for at least about 10 days in culture medium comprising about 10 ng/ml rhEGF, about 20 ng/ml FGF-2, and about 20 ng/ml NGF.

Purified, concentrated or enriched cells may be administered as a pharmaceutically or physiologically acceptable preparation or composition containing a physiologically acceptable carrier, excipient, or diluent, and administered to the tissues of the recipient organism of interest, including humans and non-human animals. The stem cell-containing composition may be prepared by resuspending the cells in a suitable liquid or solution such as sterile physiological saline or other physiologically acceptable injectable aqueous liquids. The amounts of the components to be used in such compositions can be routinely determined by those having skill in the art.

The VSELs or compositions thereof may be administered by placement of the stem cell suspensions onto absorbent or adherent material, i.e., a collagen sponge matrix, and insertion of the stem cell-containing material into or onto the site of interest. For injectable administration, the composition is in sterile solution or suspension or may be resuspended in pharmaceutically- and physiologically-acceptable aqueous or oleaginous vehicles, which may contain preservatives, stabilizers, and material for rendering the solution or suspension isotonic with body fluids of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

### Examples

The experimental results provided demonstrate that human VSELs engraft, survive, and differentiate into retinal or neuroectodermal cells when transplanted into mouse retina.

G-CSF Mobilized Human Peripheral Blood Mononuclear Cells.

Healthy human donors were mobilized by subcutaneous injection of 480 μg human G-CSF (Amgen, Thousand Oaks, Calif.) daily for 3 days. Mobilized donors were apheresed on a Cobe Spectra blood cell separator for 3-4 hours to obtain mononuclear cells.

Size-Based VSEL Enrichment by Elutriation.

Between 2 and 2.5×10¹⁰ fresh apheresis mononuclear cells were loaded onto the Elutra® cell separation system (Caridian BCT) at a concentration of 1×10⁸ cells/ml. Cells were then separated into bags in 900 ml PBS+0.5% Human Serum Albumin (HSA) media per bag using a centrifugation speed of 2,400 rpm and counterflow rates ranging from 35 ml/min to >110 ml/min Typically, five fractions were collected, with VSELs co-purifying with smaller cells in the 50 to 70 ml/min fractions.

Enrichment of CD34/133+VSELs by Immunomagnetic Selection.

Elutra fractions with VSELs were further enriched using immunomagnetic CD34 and CD133 beads (Miltenyi Biotec). 1×10⁸ cells were stained with 50 μl CD133 and 100 μl CD34 magnetic beads following the manufacturer's instructions, then washed and loaded onto an AutoMACS cell separator (Miltenyi Biotec) to enrich for the CD133⁺/CD34⁺ VSEL population. Typically, about 1 in 1,000 cells at this stage were Lin⁻CD45⁻ CD133/34⁺ VSELs.

Purification and Analysis of VSELs by Flow Cytometry.

Cells were stained for lineage markers using the following FITC-conjugated murine anti-human antibodies: anti-CD2, CD3, CD14, CD66b, CD24, CD56, CD16, CD19, CD235a and CD41. Cells were also stained with the viability dye 7-AAD and with anti-CD45-PE and a combination of CD133-APC and CD34-APC. For analysis and counting of VSELs, the nuclear dye DRAQ5 was also added to the staining cocktail. Staining was performed in PBS containing 0.5% HSA for 30 minutes on ice. VSELs were then analyzed on a Gallios flow cytometer (Beckman Coulter), or purified by sorting for Lin-CD45-CD133/CD34+ small cells (5 to 9 μm) using a MoFlo XDP cell sorter (Beckman Coulter). Both FACS-purified or MACS-enriched were frozen in PBS with 5% human albumin and 5% DMSO until needed for injection.

VSEL Transplantation into SCID Mice.

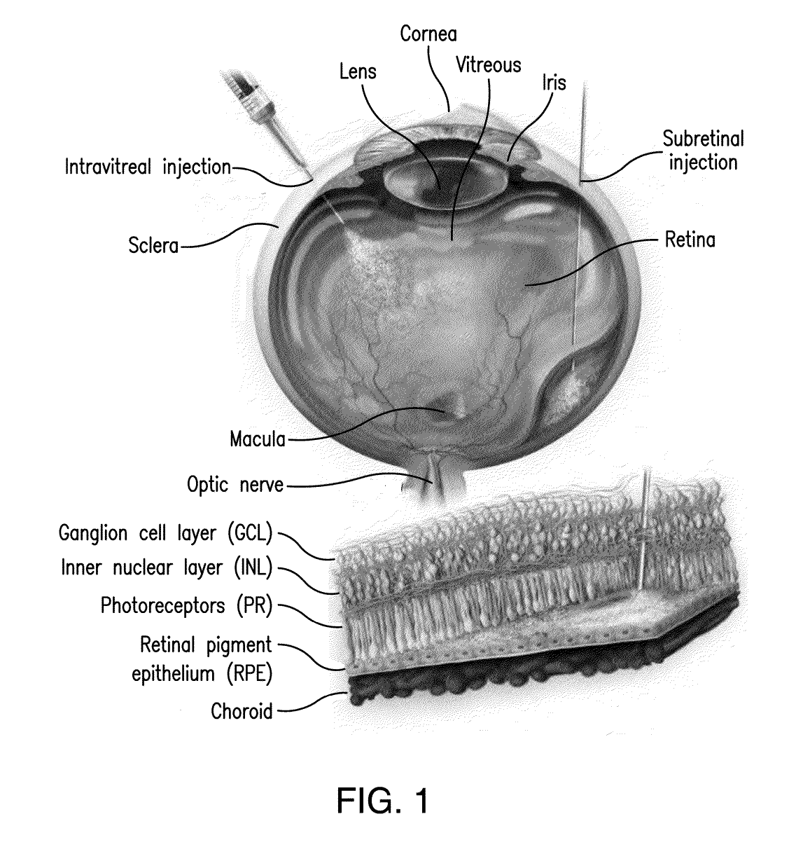

We examined two models of retinal disease in SCID mice (FIG. 1): one in which retinal ganglion cells die as in glaucoma (toxin injection followed by intravitreal injection) and one in which photoreceptors die as in macular degeneration, retinitis pigmentosa and retinal detachment (retinal detachment followed by subretinal space injection). For intravitreal injection studies, FACS-purified VSELs were labeled with the red fluorescent dye PKH-26 and injected into the vitreal space of anesthetized mice (approximately 2,000 VSELs per mouse) that had been pre-treated with the retinal ganglion cell toxin NMDA. For subretinal injection studies, enriched VSELs (preparations ranged from 0.1% to 11% pure) were labeled with the red fluorescent dye PKH26 and injected into the subretinal space (approximately 10,000 cells per mouse), creating a retinal detachment at the site of injection.

Analysis of Ocular Sections by Immunofluorescence Microscopy.

4 weeks after injection, mice were euthanized and enucleated. Eyes were fixed in 4% parafoimaldehyde, cryoprotected with sucrose in phosphate buffer, and sectioned at 8 on a cryostat. Tissue sections were immunostained for markers of retinal stem and developing progenitor cells such as Ki67, Nestin and PAX6, markers of neuro-ectodermal cells such as MAP2 and beta-3-tubulin, and for the early photoreceptor marker recoverin as well as for the photoreceptor marker rhodopsin. Some sections were also stained for the human nuclear marker SRP14 to determine the presence of human cells that had lost the PKH-26 label. Sections were examined by conventional and confocal microscopy.

The following cell survival studies examined two models of retinal disease in SCID mice: one (retinal toxin followed by intravitreal injection) in which retinal ganglion cells die, modeling diseases such as glaucoma, and the other (retinal detachment followed by subretinal space injection) in which photoreceptors die, modeling conditions such as macular degeneration, retinitis pigmentosa and retinal detachment. Both purified and enriched populations of Lin⁻CD45⁻CD34/CD133⁺ human VSELs can be easily labeled with PHK-26 and traced specifically after transplantation by fluorescence microscopy. Transplanted cells survived well in SCID mice for up to 4 weeks. Intravitreally transplanted cells tended to adhere to the inner limiting membrane and did not migrate into the retina, whereas cells injected into the subretinal space showed evidence of migration as far as the outer nuclear layer of photoreceptors. Cells transplanted into the subretinal space also showed evidence of differentiation along the retinal lineage.

Human VSELs Survive in the SCID Mouse Ocular Microenvironment Following Intravitreal Injection.

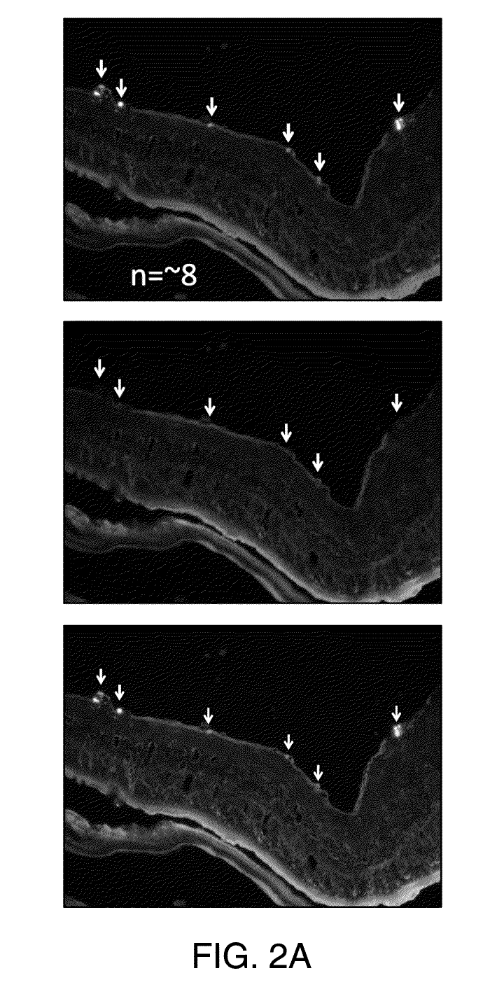

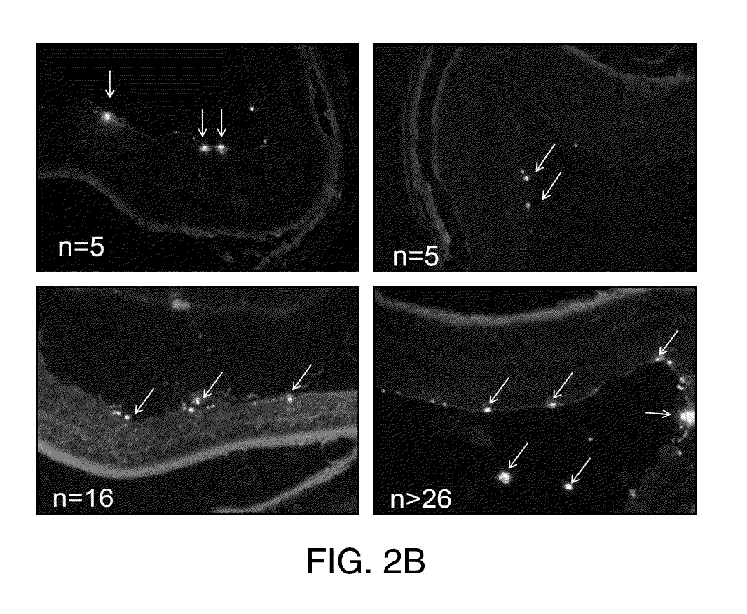

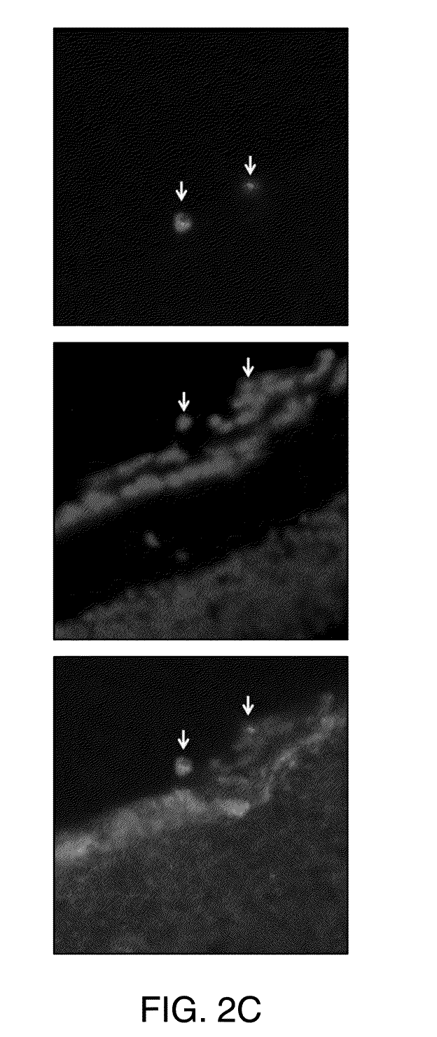

Frozen VSELs were thawed and labeled with PKH-26, then intravitreally injected into SCID mice pre-treated with the retinal ganglion cell toxin N-methyl D-aspartate (NMDA). After 4 weeks post-transplantation, 80 to 100 sections were cut and counted from each eye, about 600 sections in all, by confocal fluorescent microscopy (FIG. 2). The average number of PHK-positive cells was 13 per section, with an estimated survival rate of greater than 30%.

Enriched VSELs Injected into the Subretinal Space Survive and Migrate into the Retina.

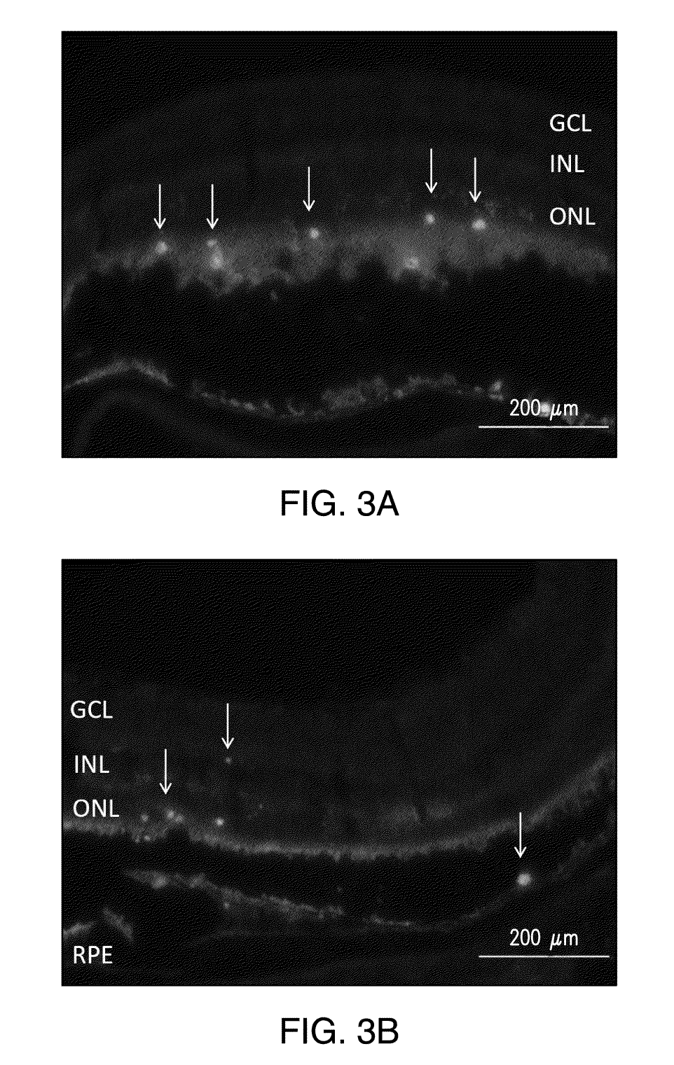

Frozen enriched VSELs were thawed and labeled with PKH-26, then injected into the subretinal space of SCID mice, creating a retinal detachment at the site of injection. After 2 and 4 weeks post-transplantation, eyes were sectioned and examined for the presence of PKH-26+ cells (FIG. 3). PKH-26+ cells were observed both in the sub-retinal space and within the retina, indicating that the cells had begun to migrate.

Some of the Transplanted Cells Display Markers of Retinal Differentiation.

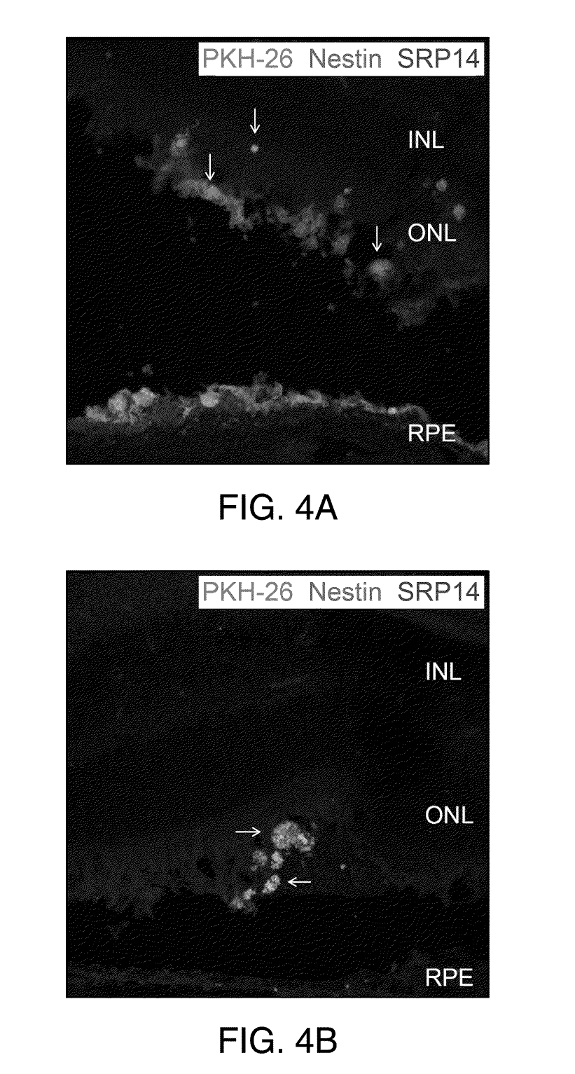

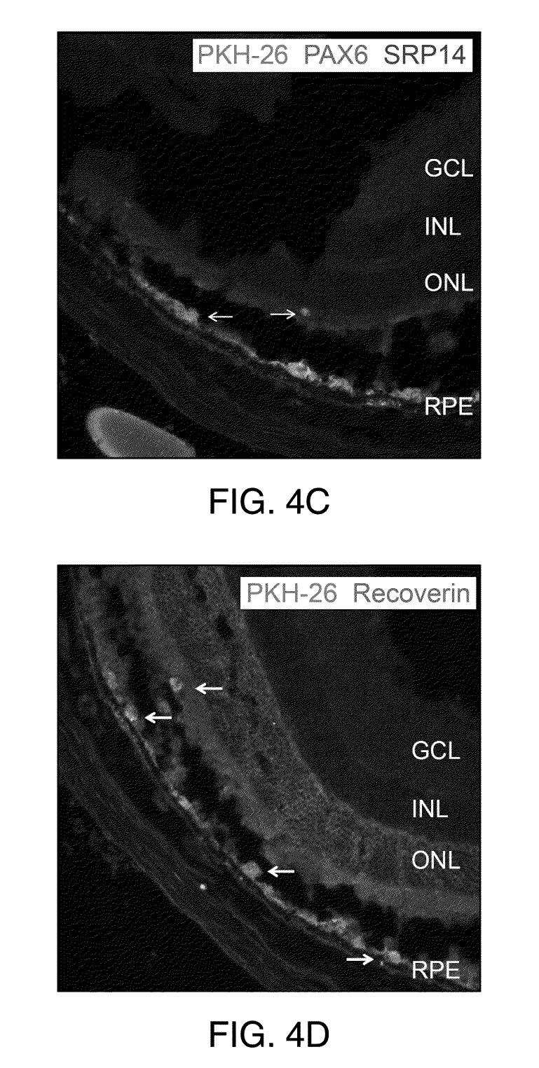

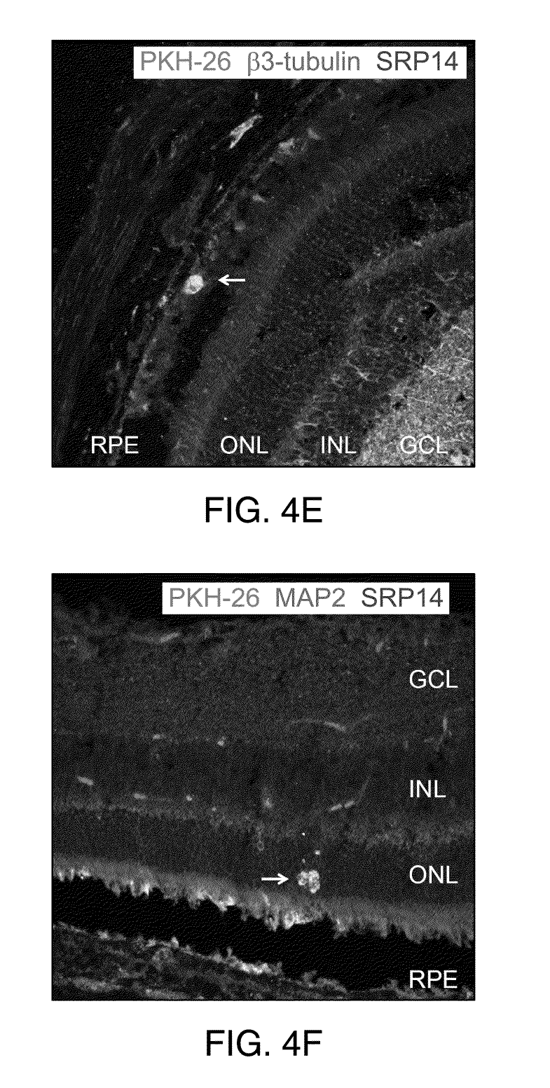

Sections were examined for evidence of differentiation of the transplanted cells along the retinal lineage by staining for markers of retinal stem and developing progenitor cells (intermediate filament protein Nestin and transcription factor PAX6), for markers of neuro-ectodermal cells (MAP2 and b3-tubulin, which are both cytoskeletal markers) and the early photoreceptor marker recoverin (a cytoplasmic protein). In addition, sections were stained with human-specific antibodies to ubiquitous nuclear (HuN) and cytoplasmic (SRP14) proteins to identify transplanted cells that may have lost the PKH-26 marker. As seen in FIG. 4A, a number of cells show co-localization of PKH-26 and SRP14 staining, and additional cells show single staining for SRP14, indicating that these are human cells that had lost the PKH-26 stain.

Some of the Transplanted Cells Display Markers of Retinal Differentiation.

Sections were examined for evidence of differentiation of the transplanted cells along the retinal lineage by staining for markers of retinal stem and developing progenitor cells (intermediate filament protein Nestin and transcription factor PAX6), for markers of neuro-ectodermal cells (MAP2 and β3-tubulin, which are both cytoskeletal markers) and the early photoreceptor marker recoverin (a cytoplasmic protein). In addition, sections were stained with human-specific antibodies to ubiquitous nuclear (HuN) and cytoplasmic (SRP14) proteins to identify transplanted cells that may have lost the PKH-26 marker. As seen in FIG. 4A, a number of cells show co-localization of PKH-26 and SRP14 staining, and additional cells show single staining for SRP14, indicating that these are human cells that had lost the PKH-26 stain.

We claim:

1. A method for treating or ameliorating a retinal disease of a mammal comprising administering an effective amount of a composition comprising very small embryonic-like stem cells (VSELs) into an eye of the mammal.

2. The method of claim 1, wherein the VSELs comprise CD45⁻/lin⁻/CD34⁺, or CD45⁻/lin⁻/CD133⁺, or CD45⁻/lin⁻/CD34⁺/CD133⁺.

3. The method of claim 2, wherein the VSELs express one or more of SSEA-4, Oct-4, Rev-1, and Nanog.

4. The method of claim 1, wherein the VSELs are further enriched for CXCR4 expression.

5. The method of claim 1, wherein the VSELs are further enriched for staining by a nuclear dye.

6. The method of claim 1, wherein the nuclear dye is DRAQ5

7. The method of claim 1, wherein the VSELs are administered subretinally.

8. The method of claim 1, wherein the VSELs are administered intravitreally

9. The method of claim 1, wherein the VSELs are administered in a suspension or matrix.

10. The method of claim 1, wherein the number of VSELs administered is from 20 to 200.

11. The method of claim 1, wherein the number of VSELs administered is from 200 to 1,000.

12. The method of claim 1, wherein the number of VSELs administered is from 1,000 to 5,000.

13. The method of claim 1, wherein the number of VSELs administered is from 5,000 to 50,000.

14. The method of claim 1, wherein the retinal disease is macular degeneration or retinitis pigmentosa.

15. The method of claim 1, wherein the mammal is a human.

16. The method of claim 1, wherein the VSELs are autologous VSELs.

17. The method of claim 1, wherein the VSELs are allogenic VSELs.

18. The method of claim 1, wherein the VSELs are human VSELs.

19. The method of claim 1, wherein at least a proportion of the administered cells engraft and express one or more markers of neural or retinal differentiation.

20. The method of claim 19, wherein the markers of neural or retinal differentiation are Nestin, PAX6, rhodopsin, recoverin, β3-tubulin and MAP2.

21. The method of claim 1, further comprising administering to the mammal a substance that stimulates differentiation of said VSELs into photoreceptors cells.

22. The method of claim 1, further comprising administering to the mammal, a substance that stimulates differentiation of said VSELs into neurons.

23. The method of claim 1, wherein the composition comprises cells that are at least 50% VSELs.
